(19) United States
(12) Patent Application Publication
(10) Pub. No.: US 2018/0276611 A1
(43) Pub. Date: Sep. 27, 2018

(54) Secure Network Based Order Confirmation, Transportation, and Delivery Processes Utilizing Logistics Automation

(71) Applicant: Web Access, LLC., Calverton, NY (US)

(72) Inventors: Bryan Dromerhauser, Bay Shore, NY (US); Nathaniel T. Bradley, Tucson, AZ (US); Joshua S. Paugh, Tucson, AZ (US)

(73) Assignee: Web Access, LLC., Calverton, NY (US)

(21) Appl. No.: 15/928,685

(22) Filed: Mar. 22, 2018

Related U.S. Application Data
(60) Provisional application No. 62/475,580, filed on Mar. 23, 2017.

(51) Int. Cl. G06Q 10/08; G16H 20/13; G06F 17/30

(52) U.S. Cl. CPC G06Q 10/0833; G06F 17/30864; G16H 20/13

(57) ABSTRACT

One embodiment provides a system, method, platform, and server for processing orders for medication. An order for medication is received including a location of a user performing the order. Confirmation information is requested from the user. A determination is made the order requirements are met. The medications are provisioned for delivery. One or more identifiers are requested to complete the delivery to the user.

### PRIORITY

This application claims priority to U.S. Provisional Patent Application Ser. No. 62/475,580 entitled SECURE NETWORK BASED ORDER CONFIRMATION, TRANSPORTATION, AND DELIVERY PROCESSES UTILIZING LOGISTICS AUTOMATION filed on Mar. 23, 2017, the entirety of which is incorporated by reference herein.

### BACKGROUND

### I. Field of the Disclosure

The illustrative embodiments relate to medication delivery. More specifically, but not exclusively, the illustrative embodiments relate to secure network based order confirmation, transportation, and delivery utilizing network logistics.

### II. Description of the Art

Many individuals need, require, or want various forms of medication to better their lives. For example, may individuals must travel to a dispensary, pharmacy, kiosk, or other location to retrieve their medications or other items. Thus, significant time, resources, and effort is spent to find, purchase, and retrieve desired medications.

### SUMMARY OF THE DISCLOSURE

The illustrative embodiments provide a system, method, apparatus, platform, and server for processing orders for medication. An order for medication is received including a location of a user performing the order. Confirmation information is requested from the user. A determination is made the order requirements are met. The medications are provisioned for delivery. One or more identifiers are requested to complete the delivery to the user.

Another embodiment provides a system for managing orders of medication. The system includes a platform managing the orders as received. The system also includes one or more databases in communication with the platform storing information associated with the orders. The system also includes one or more networks in communication with the platform for sending and receiving communications associated with the orders. The platform receives the order for medication including a location of a user performing the order, requests confirmation information from the user, determines whether order requirements are met, provisions the medications for delivery, and requests one or more identifiers to complete the delivery to the user.

### BRIEF DESCRIPTION OF THE DRAWINGS

Illustrated embodiments are described in detail below with reference to the attached drawing figures, which are incorporated by reference herein, and where:

FIG. 1 is pictorial representation of an order environment in accordance with an illustrative embodiment;

FIG. 2 is a block diagram of an order platform in accordance with an illustrative embodiment;

FIG. 3 is a flowchart of a process for processing a transaction in accordance with an illustrative embodiment;

FIG. 4 is a flowchart of a process for delivering medication in accordance with an illustrative embodiment; and

FIG. 5 is a pictorial representation of a computing system in accordance with an illustrative embodiment.

### DETAILED DESCRIPTION OF THE DISCLOSURE

The description that follows includes exemplary systems, methods, devices, techniques, instruction sequences and computer program products that embody techniques of the present inventive subject matter. However, it is understood that the described embodiments may be practiced without these specific details. In other instances, well-known instruction instances, protocols, structures, components, and techniques have not been shown in detail in order not to obfuscate the description. The illustrative embodiments are meant to be combined across Figures, claims, and embodiments regardless of natural or artificial restrictions or limitations imposed upon the content. The description and language herein, is applicable across all of the Figures regardless of Figures, call outs, or so forth. The users of the illustrative embodiments may be referred to as users, patients, individuals, purchasers, or so forth.

The illustrative embodiments, provide a system, method, and apparatus for performing an order transaction. In one embodiment, the order may include medications, drugs, items, or products. The order may be tracked through all stages of processing (e.g., receipt, provisioning, fulfillment, delivery, verification, follow-up, etc.). For example, the order may include medical marijuana that is delivered according to state, county, local, and/or country or federal laws. Careful and meticulous processing of the order may be required for applicable certifications, legal requirements, best practices, industry standards, and so forth.

User information, driver information, purchaser information, and/or any number of identifiers may be utilized to securely deliver the medication or purchased items. In one embodiment, a secure storage unit integrated with a vehicle may ensure that the medication is safely and accurately delivered. The deliveries may occur through autonomous or user controlled vehicles. Information related to the order (e.g., transaction, mapping, driver qualifications, driver location, expected delivery time, location, medication availability, delivery confirmation, etc.) may be managed for a number of different parties. In one embodiment, the purchaser may be required to provide a passcode, serial number, QR code, confirmation code, or other information to complete the purchase. The illustrative embodiments may be implemented with driver, drone, or other delivery systems and services, such as Uber, Lyft, Door Dash FedEx, UPS, USPS, DHL, or other similar services as are known in the art.

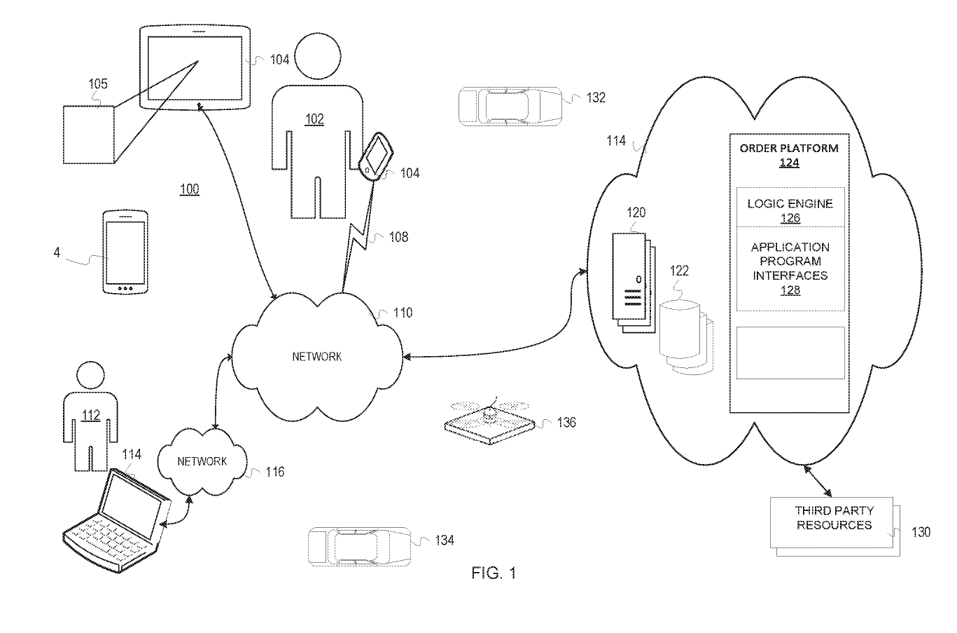

FIG. 1 is pictorial representation of an order environment 100 in accordance with an illustrative embodiment. The order environment 100 may include any number of users, systems, devices, structures, roads and pathways, networks, connections, signals, and so forth. In one embodiment, the order environment 100 may include a user 102, a wireless device 104, a wireless signal 108, a network 110, a user 112, a computing device 114, a network 116, a cloud network 118, servers 120, databases 122, order platform 124, logic engine 126, third party resources 130, and delivery vehicles 132, 134, and 136.

In one embodiment, the user 102 may represent an individual, party, organization, or entity submitting an order. The order may be entered by the user 102 the wireless device 104 and sent to the cloud network 118 through the network 110. Any number of wireless, wired, or other forms of electronic signals may be utilized to communicate the order.

Similarly, the user 112 may utilize the computing device 114 to submit the order to the cloud network 118 through the network's 116, 110. As shown, the devices utilized by the user's 102, 112 may represent any number of computing or communications devices, such as smart phones, laptops, desktop computers, gaming devices, wearable devices (e.g., smart watches, medical bracelets, wireless earpieces, smart clothing, etc.), media systems, displays, or so forth.

The cloud network 118 may represent any number of systems, devices, equipment, platforms, or components for processing the order. For example, the cloud network 118 may represent a system or cloud system as is known in the art and is herein described. The components of the cloud network 118 may operate jointly or independently as needed. In one embodiment, the servers 120 of the cloud network 118 may receive incoming orders to perform processing. The servers 120 may also store the orders and related information in the databases 122. In one example, the cloud network 118 may be utilized by a single retailer, manufacturer, group, entity, or individuals. In other examples, the cloud network 118 may be operated for a plurality of different individuals, entities, or other parties.

The order platform 124 may represent one or more of the servers 120 or a separate platform that processes the orders. The order platform 124 may represent one or more stand-alone or networked devices that are off-the-shelf or customized for the order requirements. The order platform 124 may utilize the logic engine 126 to process the orders. For example, the logic engine 126 may represent digital logic, software, algorithms, scripts, modules, sets of instructions, or so forth. The order platform 124 may also include any number of Application Program Interfaces (APIs) 128 for interfacing with different mobile applications, operating systems, kernels, or third party systems, equipment, or devices. The cloud network 118 may also access the third party resources 130. The third party resources 130 may represent any number of systems, devices, databases, lists, users, or other applicable information that may be relevant to the lawful and efficient processing of the order. For example, the third-party resources 130 may represent medical professionals, health insurance providers, legal databases, state or federal resources, and so forth. The third party resources 130 may also represent compliance databases (e.g., government, regulatory, industry, etc.), safety databases, block chain systems, cryptocurrency systems, or other systems, equipment, database, or devices that may be accessed, updated, or so forth.

The order environment 100 and vehicles 132, 134, 136 may utilize any number of sensors to manage and track orders. The sensors may include beacons, radio frequency identification tags, chips, circuits, serial numbers, bar codes, or so forth.

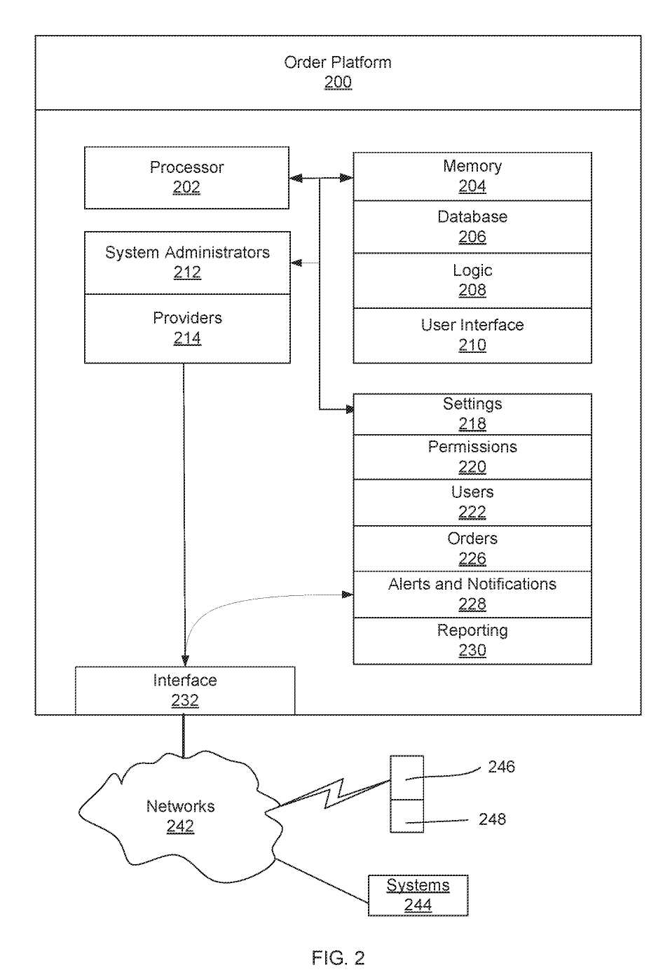

FIG. 2 is a block diagram of an order platform 200 in accordance with an illustrative embodiment. In one embodiment, the order platform 200 may include a processor 202, a memory 204, a database 206, logic 208, a user interface 210, system administrators 212, providers 214, settings 218, permissions 220, users 222, orders 226, alerts and notifications 228, reporting 230, and an interface 232. The order platform 200 is one embodiment, of the order platform 124 of FIG. 1 in accordance with an illustrative embodiment.

The order platform 200 may communicate with networks 242, systems 244, and wireless devices 246 utilizing applications 248. In one embodiment, the order platform 200 is a system, computing device, or group of networked or distributed computing devices. The order platform 200 may have any number of hierarchical users including, system administrators, service providers, delivery people, and other similar users.

In one embodiment, the processor 202 is circuitry or logic enabled to control execution of a set of instructions. The processor 202 may be microprocessors, digital signal processors, application-specific integrated circuits (ASIC), central processing units, or other devices suitable for controlling an electronic device including one or more hardware and software elements, executing software, instructions, programs, and applications, converting and processing signals and information, and performing other related tasks. The processor 202 may be a single chip or integrated with other computing or communications elements.

The memory 204 is a hardware unit, component, device, or recording media configured to store data for subsequent retrieval or access at a later time. The memory 204 may be static or dynamic memory. The memory 204 may include a hard disk, random access memory, cache, removable media drive, mass storage, or configuration suitable as storage for data, instructions, and information. In one embodiment, the memory 204 and processor 202 may be integrated. The memory may use any type of volatile or non-volatile storage techniques and media.

In one embodiment, the database 206 may be utilized to store data 226. The data 226 may include information and data related to the orders received, processed, provisioned, delivered, and otherwise managed. The data 226 may also include information relevant to inventory management, tracking, and processing. The data 226 may also store information relevant to multiple suppliers, users, manufacturers, retailers, or so forth. The database 206 may include a database management system such as SQL (and variations), Access, Oracle, DB2, or other developing systems. In one embodiment, the interface 232 may include a transceiver configured to both transmit and receive communications for the order platform 200. The communications may represent wired or wireless communications. The networks 242, systems 244, wireless devices 246 and applications 248 may represent all or a portion of the transaction system 100 of FIG. 1.

In one embodiment, information, settings, and data utilized by the order platform 200 is saved or duplicated to one or more redundant systems for backup, failover, or network processing utilizing any number of backup standards, formats, and protocols.

In one embodiment, system administrators 212 may monitor, control, or manage the orders received and processed by the order platform 200. The system administrators 212 may be allowed to manage the setup of information, settings, and modules for the provider 214. In one embodiment, the system administrators may be prevented from seeing specific types of data (e.g., user financial data, credit cards, passwords, etc.). The providers 214 may represent manufacturers, pharmacies, distribution groups, delivery providers, growers, drivers, medical professionals, and so forth.

The user interface 210 may be represented by programs, applications, secure access protocols or systems made available through the order platform 200, such as the user interface 210, or alternatively, the applications 248, or the systems 244. The user interface 210 may present a dashboard, site, or portal for managing the orders and described information and data. In one embodiment, the order functions of the user interface 210 may be accessed through a desktop application interface or webpage. In one embodiment, the user interface 210 may display any number of icons, images, drop-down menus, videos, hyperlinks, internal links, scroll wheels, tabs, Windows, or other selection or display components.

The system administrators 212 may have the capacity to add, edit, archive, and delete the providers 214 based on performance, regulations, laws, certifications, qualifications, jurisdiction, location, errors, mistakes, or so forth. In one embodiment, the logic 208 is the software, programming, hardware, logic, or instructions for processing the orders as is herein described. The logic 208 may also be integrated with other components of the order platform (e.g., processor 202, memory 204, database 206, user interface 210, etc.). In one embodiment, the logic 208 may generate status information with regard to the orders. For example, the status information may indicate when orders are received, how and when they are processed, confirmation information, funds processed, provider selection, delivery status, delivery confirmation, order feedback, and so forth.

The order platform 200 stores and provides access to every order. As a result, the system administrators 212 and the providers 214 may log information associated with their associated orders. System administrators 212 may also post public notes, comments, alerts, notifications, associated with data being recorded by the order platform 200 through the individual caregiver accounts. Applicable policies rules, laws, industry practices, and so forth may also be made available to individuals, groups, businesses, users, and others that may access the order platform 200 or interfaces that interact with the order platform 200. For example, the order platform 200 may be accessible through a webpage, mobile applications, telephonic systems, and so forth. The users may utilize the applications 248 (e.g., Android, iOS, etc.) to communicate with the user interface 210 to make, view, and edit orders 226. The user interface may provide an easy to navigate and user friendly interface with quick search utilities and filters.

The communications with the order platform 200 are secured for sensitive electronic protected health information, financial transactions, and to comply with applicable laws, such as HIPAA, privacy laws, financial regulations, and so forth. Communications with the order platform 200, whether in real-time or file transfers, may be encrypted or utilize other secure data transmission protocols (i.e., SSL, https, security certificates, etc.). The order platform 200 may also be configured to communicate with any number of databases or systems maintained by the system administrators, such as electronic databases. The applications 248 may process orders and data even if one of the networks 242 is not currently available or if the wireless devices 246 and the systems 244 are offline. Synchronization methods and modes may be utilized as necessary.

The settings 218 and permissions 220 may specify information for the users (e.g., consumers making orders), system administrators 212, providers, and so forth. The users 222 may include accounts, user profiles (e.g., age, sex, health issues, insurance, medication limitations, legal status, etc.), payment information, and other application information for the ordering users that access the order platform 200. The orders 226 indicate information and data applicable to past and current orders. The orders 226 associated with specific users 222, providers 214, and system administrators 212 may be accessed as allowed.

In one embodiment, the alerts and notifications 228 may provide alerts, notifications, or other relevant information to the applicable users 222, providers 214, system administrators 212, applications 248, wireless devices 246 or so forth. The alerts and notifications may include program based messages, audio messages, video messages, text messages, in-app messages, emails, and so forth.

In one embodiment, the reporting 230 may be utilized to communicate order information which may include statistics, charts and graphs, transaction information, delivery information, and so forth. In one embodiment, the reports of the reporting 230 may be based on users, location, service provider, delivery vehicle/driver, region, medications, date ranges, and so forth. The reports are configured to be customizable to track and display a variety of information and data available through the order platform 200. For example, a message listing the orders for the day, the purchasers address, quantity, and type of order may be communicated through an alert or other message.

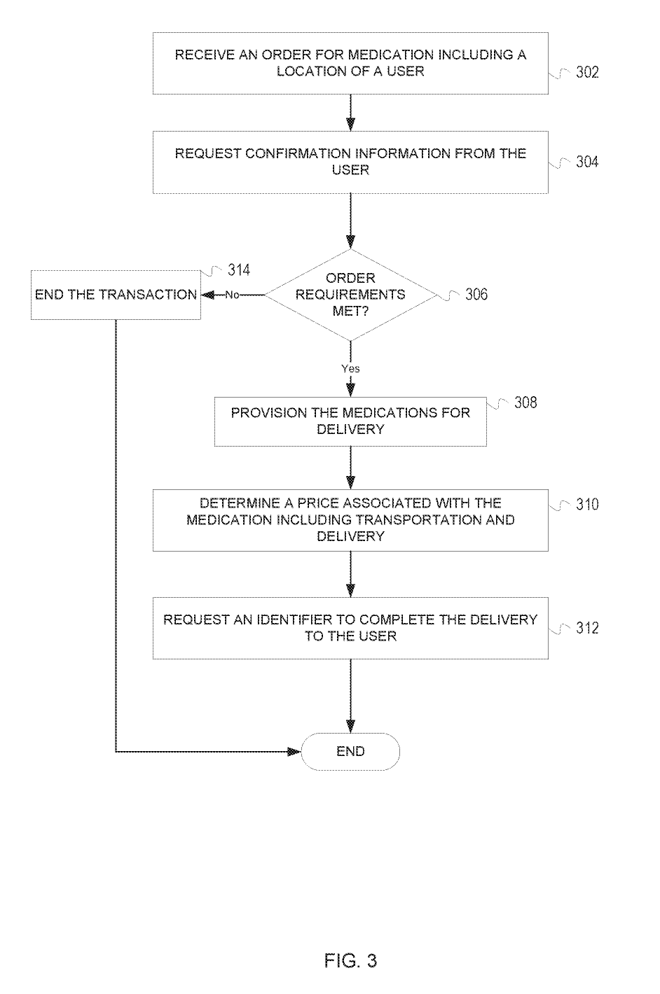

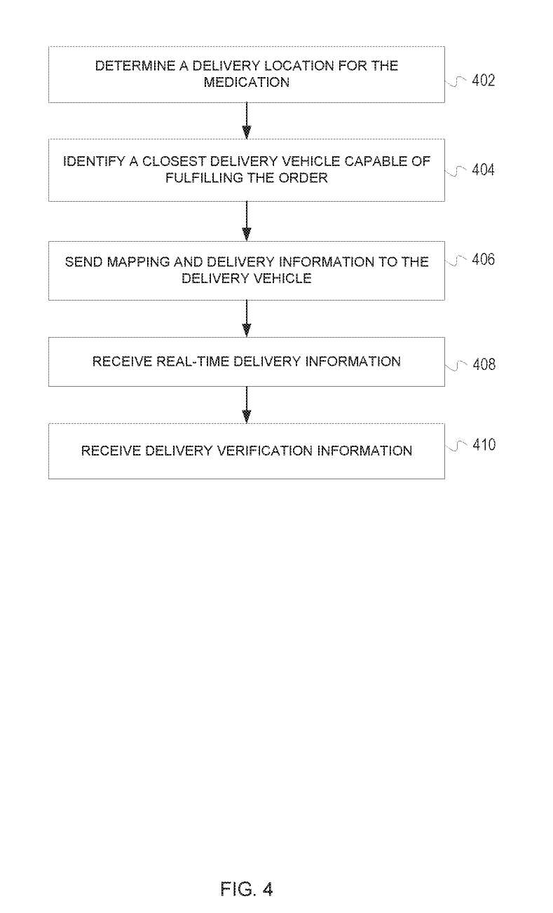

FIG. 3 is a flowchart of a process for processing a transaction in accordance with an illustrative embodiment. In one embodiment, the process of FIGS. 3 and 4 may be performed by a system, platform or environment, such as the order environment 100 or order platform 124 of FIG. 1. For example, the system may include any number of mobile or wireless devices, computing devices, networks, processing systems (e.g., cloud networks, servers, databases, artificial intelligence, intelligent devices, etc.), delivery vehicles (e.g., manned, autonomous, etc.), and so forth.

In one embodiment, the process of FIG. 3 may begin by receiving an order for medication including a location of a user (step 302). The medication may include any number of prescription, herbal, medicinal, or other lawful medications. For example, in some states, the medications may include marijuana. The order may be received through any number of computing or communications devices associated with the ordering user. For example, the order may be received through a mobile app, website or online portal, dial in system, text message ordering system, or so forth. The order may also include location, delivery, or address information associated with the user. The location information may be determined utilizing a global positioning system (GPS), wireless triangulation, IP address mapping, beacons, user provided address or delivery point, or location-based services. As a result, the location information may be determined utilizing any number of automated or user provided processes or information. Historical delivery information may also be utilized.

Next, the system requests confirmation information from the user (step 304). During step 304, the system may verify that the user is authorized to receive or take the medication. For example, the user's legal age, health status, legal status (e.g., criminal status, limitations on medications, etc.), insurance, primary care physician, and/or other applicable information that may permit or deny access to the medication. The system may access any number of third-party systems, databases, or approvals in order to perform the confirmation. For example, the user's age may be independently verified using a state or federal driver's license database. In another example, a medical professional associated with the user may be contacted to verify that the medication is authorized for the user as ordered (e.g., quantity, type, delivery method, etc.).

Next, the system determines whether the order requirements are met (step 306). The order requirements may include any number of business, legal, best practices, or industry standards, rules, or laws. In one embodiment, the order requirements are confirmed and verified during step 304. The requirements may include any number of medical, legal, and other questions that require answering or verification. For example, one or more questions may be posed to the user, such as “Are you John Smith, Jr.?”, “Is the quantity of medication you received correct?”, “Are you authorized to receive this medication?”, “Do you have any allergies that would you from taking this medication?”, “Have you consulted a Doctor or a Pharmacist with any questions you have regarding this medication? Do you need to?”, and any number of other questions. Any number of text based, toggle, drop down, or other questions and answers may be utilized. User input, feedback, instructions, and/or commands may be received textually (e.g., words, letters, numbers, etch), tactilely, verbally, gesturally, or based on other user input.

In response to determining the order requirements are met during step 306, the system provisions the medications for delivery (step 308). During step 308, the system may select a nearest dispensary, pharmacy, delivery vehicle, or other repository that may have the medication in a type, quality, and quantity sufficient to fill the order. The system may utilize logic, algorithms, applications, data, or other information to provision the medications. In one embodiment, any number of mapping and inventory systems or databases may be utilized to track the medications. For example, the system may determine that the specified product and quantity are on-board a nearest vehicle to fulfill the order. In one embodiment, the nearest delivery vehicle may be selected based on availability, proximity, capability, certifications, safety, and so forth. The delivery vehicles may include any number of manned (e.g., cars, buses, delivery vehicles, Weed Wagons, commercial delivery services, etc.) or autonomous systems (e.g., flying drones, driving drones, pneumatic systems, etc.). For example, the area may be required to allow drone deliveries as stipulated by the FAA, transportation administration, or other applicable country, federal, or state entities. During step 308, the delivery route may be calculated, uploaded, or otherwise communicated as needed.

Next, the system determines a price associated with the medication including transportation and delivery (step 310). The price may include the price for the medication, processing costs, delivery costs, taxes, and so forth. In one embodiment, the price may be sent to the ordering user for acceptance before the medication is delivered. In another embodiment, a price range may be estimated for the order and the user may pre-accept the price range to complete the order. In one embodiment, during step 310, the user may be charged for the order utilizing an account, credit card, payment service (e.g., PayPal, Google Wallet, Apple Pay, Venmo, Dwolla, electronic deposit, etc.), insurance card, cash, wire transfer, or so forth. In some embodiments, a premium, upcharge, or discount may be charged or applied for time of day, available delivery people, product availability, and so forth.

Next, the system requests an identifier to complete the delivery to the user (step 312). In one embodiment, the identifier is a unique code that was sent to the user (or a device associated with the user) during the order process. In one embodiment, the identifier is associated with a user profile/account for the user. The user profile may be updated by the user at any time utilizing a browser, web application, or other user interface to the system herein described. The identifier may also include user identifiers, such as driver's licenses, passports, pin number, password, alphanumeric code, health insurance information, student identification, immigration status card, and so forth. The identifier may also include one or more biometric indicators associated with the user. The identifier may represent a confirmation code that is required to retrieve the order from a drive or a secure storage device attached to or integrated with the vehicle. For example, the identifier may represent a computer readable code, barcode, UPC symbol, password, QR code, image, or other identifier that is unique to the specified order and user. Once the identifier is verified, the delivery may be completed to the user.

In one embodiment, items of inventory, such as medications, may be stored within secure lockboxes, compartments, or containers that may be part of a secure vending machine, storage unit, or other container located onboard each transportation and delivery vehicle. As noted, the identifier may be required to access items stored within the secure container to perform authenticated transactions, prevent thefts, prevent substance abuse, and so forth. The identifier may be utilized to deliver or open the secure container for the user. For example, the medication may be stored in a receptacle of the vehicle and then may be delivered in a hemp based envelope with an embedded readable code. In one embodiment, the readable code may be utilized by the vehicle, driver, or associated vending machine to locate and release the specified product order associated with the readable code. The identifier may represent a unique readable code embedded within, attached to, or printed on the delivery envelope, package, bottle, bag, container, or delivery receptacle. The identifier may be communicated to any number of communications or computing devices, such as smart phones, wearables (e.g., watches, headphones, etc.), vehicle systems, and media devices.

In one embodiment, other information, data, or content may also be associated with the identifier, order, or other identifying portions of the transaction. For example, the information, data, or content may include metadata, de-identified end-user data, instructions for use, delivery or consumption, warnings (e.g., medical, legal, health insurance, etc.), multimedia (e.g., 3D content, video, virtual reality, preprogrammed responses, etc.).

In one embodiment, the order may only be delivered at a specified time, location, or based on specified circumstances or settings. The specified information may prevent access to the secure container that is not allowed, illegal, or unwanted. The security containers may include any number of locks, passwords, and secure identifiers. For example, the identifier of step 312 may represent identifiers from the user as well as identifiers provided by the manufacturer, seller, system, vehicle, driver, or so forth. In another embodiment, the delivery vehicle or the secure container may be configured to contaminate, destroy, or taint the order making it unusable for unauthorized parties that try to improperly access medications, items, or so forth.

Sensors within the delivery vehicle or associated secure unit may communicate with the system regarding inventory levels for resupplying and restocking the vehicle. For example, in response to determining the inventory levels, a vehicle may be sent to a restocking facility or supplemental deliveries may be made to the delivery vehicle.

If the order requirements are not met during step 306, the system ends the transaction (step 314). During step 314, any number of communications or alerts may be sent to the ordering user, delivery driver/vehicle, system administrator, authorities (e.g., police, hospital, etc.). In addition, the reasons for failure of the transaction may be recorded for subsequent reference. For example, an illegal attempt to obtain medication by a minor or felon may be noted for future reference.

FIG. 4 is a flowchart of a process for delivering medication in accordance with an illustrative embodiment. The process of FIG. 4 may also be implemented by a system and delivery vehicle.

The process may begin by determining a delivery location for the medication (step 402). The delivery location may be specified as part of the order. The delivery location may differ from a home address, billing address, or so forth as specified by the user. The delivery location may also be automatically determined utilizing location services, components, or devices of an applicable wireless device (e.g., smart phone, GPS, etc.).

Next, the system identifies a closest delivery vehicle capable of fulfilling the order (step 404). During step 404, the system may evaluate a number of potential delivery vehicles. Information, such as proximity to the ordering user, inventory (e.g., quantity, quality, type of items, etc.), certifications, driver qualifications (e.g., age, license, legal stipulations, etc.). In another embodiment, the system may identify a nearest kiosk, vending machine, or dispensary for the ordering user (to retrieve the contents of the order him/herself).

Next, the system sends mapping and delivery information to the delivery vehicle (step 406). The mapping information may be utilized to ensure that the delivery vehicle arrives at the location as soon as possible. For an autonomous vehicle the mapping information may specify an address or GPS location along with any specific driving, navigating, or flying instructions. For a manned vehicle, the mapping information may include turn-by-turn navigation data. In another embodiment, the system may send the mapping and delivery information to the ordering user to travel to the kiosk, vending machine, dispensary, or so forth. For example, the system may also provide directions for the user to reach the storage location of the medication (e.g., driving, biking, walking, etc.).

Next, the system receives real time delivery information (step 408). In one embodiment, the delivery information may include confirmation information and an identifier (e.g., FIG. 3 steps 304, 312). The delivery information may specify the location of the delivery vehicle, expected arrival time, driver status, weather conditions, medication/order status, driver identification, vehicle identification, vehicle speed and heading, and so forth. In one embodiment, all or portions of the delivery information may be displayed to the ordering user. For example, a hyperlink included in an email confirmation to the ordering user may be utilized to access the delivery information from the system (or interconnected systems, devices, equipment, components, etc.). The delivery information may also include images, video, and/or audio recordings of the ordering user retrieving or receiving the order from the vehicle, drive, or so forth. The cameras or video may be from an integrated system, from vehicle cameras, body cameras worn by the driver, a smart phone, or so forth.

Next, the system receives delivery verification information (step 410). The delivery verification information may indicate when, where, how, and to whom the order was fulfilled. The delivery verification information may be tracked for any number of business and legal purposes. The delivery verification information may also be accessible through any number of databases, websites, server/client systems, mobile applications, and so forth. The delivery verification information may also be sent to a number of relevant parties, such as user specified individuals, medical professionals, guardians, administrators, parents, health insurance providers, and so forth.

The illustrative embodiments may also utilize smart bottles or dispensers for ensuring that the medications are delivered in a safe and effective manner. Smart, secure, and/or child proof and animal proof containers may also be utilized as the end product or delivery product. The medications may also include identifiers, such as labels, micro or nano tags or trackers, serial numbers, radio frequency tags, or so forth.

In other embodiments, the system may perform other processes and methods. For example, the system may confirm that tax is paid on the order. The taxes may include state, federal, county, consumption, luxury, or other applicable taxes and fees. In one example, the taxes may be included in the order costs, delivery costs/fees, and so forth. The system may also confirm document compliance. For example, the documents may specify whether the user is authorized to receive the medication, dispensary/pharmacy, regulatory, and legal compliance, delivery person authorization, insurance information, order information, and so forth. The system may also secure delivery and innovative logistics as are herein described. The system may also create a demand index. The demand index may track the item ordered, time of day ordered, and so forth. The demand index may be utilized to anticipate future requests and deliveries. The system may also create data and analytics. The data and analytics may be associated with the order users, providers, delivery personnel, and so forth. In some embodiments, block chain technologies may be utilized to track and document every portion of the transaction.

The dispensaries or pharmacies may also have similar or compatible systems. The dispensaries may perform inventory matching, demand index tracking, strain and brand tracking, THM, and opt-in marketing. Suppliers may use the platform to track monitor, report, maintain compliance, store data, and track analytics.

All or portions of the steps of FIGS. 3-4 may be performed completely automatically or autonomously. Other steps may require user input or feedback. In some embodiments, multiple devices, systems, equipment, or components may work together to perform the processes as are herein described.

Any number of inventory sensors or tagging processes may be utilized. In one embodiment, radio frequency identification tags, chips, or circuits, inaudible tone packages, tracking chips/circuits, or other tracking components may be utilized to monitor and track group or individual items that are sold or distributed.

The illustrative embodiments may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, embodiments of the inventive subject matter may take the form of a computer program product embodied in any tangible medium of expression having computer usable program code embodied in the medium. The described embodiments may be provided as a computer program product, or software, that may include a machine-readable medium having stored thereon instructions, which may be used to program a computing system (or other electronic device(s)) to perform a process according to embodiments, whether presently described or not, since every conceivable variation is not enumerated herein. A machine readable medium includes any mechanism for storing or transmitting information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read only memory (ROM); random access memory (RAM); erasable programmable memory (e.g., EPROM and EEPROM); flash memory; or other types of medium suitable for storing electronic instructions. In addition, embodiments may be embodied in an electrical, optical, acoustical or other form of propagated signal (e.g., carrier waves, infrared signals, digital signals, etc.), or wireline, wireless, or other communications medium.

Computer program code for carrying out operations of the embodiments may be written in any combination of one or more programming languages, including an object-oriented programming language such as Java, Smalltalk, C++ or the like and conventional procedural programming languages, such as the “C” programming language or similar programming languages. The program code may execute entirely or partially on a user's wireless device, wireless earpieces, or computer, as a stand-alone software package, partly on the user's device(s) and partly on a remote computer or entirely on the remote computer or server. In the latter scenario, the remote computer may be connected to the user's computer through any type of network, including a local area network (LAN), a personal area network (PAN), or a wide area network (WAN), or the connection may be made to an external computer (e.g., through the Internet using an Internet Service Provider).

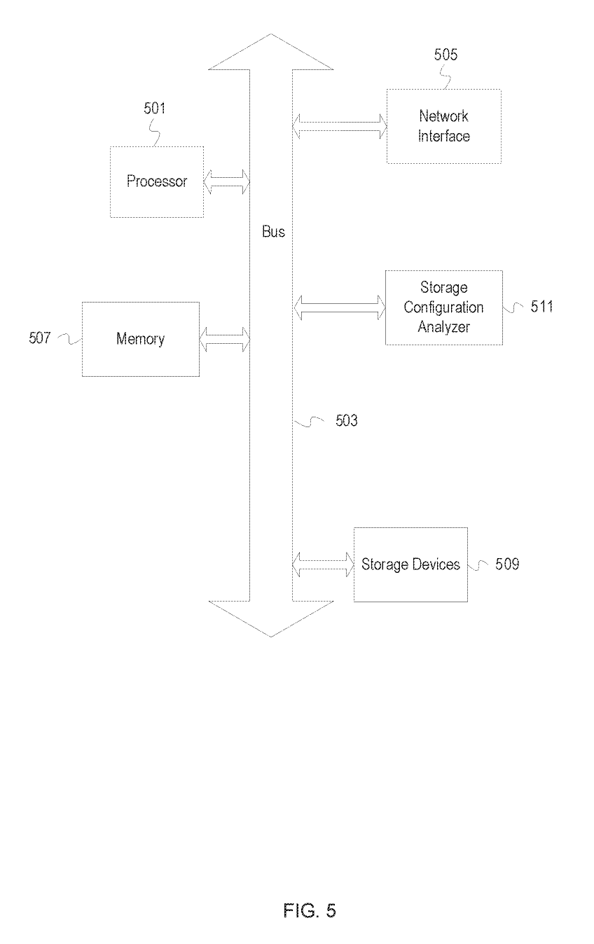

FIG. 5 depicts a computing system 500 in accordance with an illustrative embodiment. For example, the computing system 500 may represent a device, such as the order platform 124 of FIG. 1. The computing system 500 includes a processor unit 501 (possibly including multiple processors, multiple cores, multiple nodes, and/or implementing multi-threading, etc.). The computing system includes memory 507. The memory 507 may be system memory (e.g., one or more of cache, SRAM, DRAM, zero capacitor RAM, Twin Transistor RAM, eDRAM, EDO RAM, DDR RAM, EEPROM, NRAM, RRAM, SONOS, PRAM, etc.) or any one or more of the above already described possible realizations of machine-readable media. The computing system also includes a bus 503 (e.g., PCI, ISA, PCI-Express, HyperTransport®, InfiniBand®, NuBus, etc.), a network interface 606 (e.g., an ATM interface, an Ethernet interface, a Frame Relay interface, SONET interface, wireless interface, etc.), and a storage device(s) 509 (e.g., optical storage, magnetic storage, etc.).

The system memory 507 embodies functionality to implement all or portions of the embodiments described above. The system memory 507 may include one or more applications or sets of instructions for controlling content streamed to the wireless earpieces to be output by the corresponding projectors. The system memory 507 may also store an application, website, program or interface for managing orders as described herein. In one embodiment, order processing software may be stored in the system memory 507 and executed by the processor unit 502. As noted, the sharing application or software may be similar or distinct from the application or software utilized by the wireless earpieces. Code may be implemented in any of the other devices of the computing system 500. Any one of these functionalities may be partially (or entirely) implemented in hardware and/or on the processing unit 501. For example, the functionality may be implemented with an application specific integrated circuit, in logic implemented in the processing unit 501, in a co-processor on a peripheral device or card, etc. Further, realizations may include fewer or additional components not illustrated in FIG. 5 (e.g., video cards, audio cards, additional network interfaces, peripheral devices, etc.). The processor unit 501, the storage device(s) 509, and the network interface 505 are coupled to the bus 503. Although illustrated as being coupled to the bus 503, the memory 507 may be coupled to the processor unit 501.

While the embodiments are described with reference to various implementations and exploitations, it will be understood that these embodiments are illustrative and that the scope of the are not limited. In addition, the description for all embodiments and figures is applicable across figures and should not be unduly restricted. Many variations, modifications, additions, and improvements are possible.

Plural instances may be provided for components, operations or structures described herein as a single instance. Finally, boundaries between various components, operations and data stores are somewhat arbitrary, and particular operations are illustrated in the context of specific illustrative configurations. Other allocations of functionality are envisioned and may fall within the scope of the illustrative embodiments. In general, structures and functionality presented as separate components in the exemplary configurations may be implemented as a combined structure or component. Similarly, structures and functionality presented as a single component may be implemented as separate components. These and other variations, modifications, additions, and improvements may fall within the scope of the embodiments. The foregoing description has been presented for purposes of illustration and description. It is not intended to be an exhaustive list or limit any of the disclosure to the precise forms disclosed. It is contemplated that other alternatives or exemplary aspects are considered included in the disclosure. The description is merely examples of embodiments, processes or methods. It is understood that any other modifications, substitutions, and/or additions may be made, which are within the intended spirit and scope of the disclosure.

The previous detailed description is of a small number of embodiments and is not intended to be limiting in scope. The following claims set forth a number of the embodiments disclosed with greater particularity.

What is claimed is:

1. A method for processing orders for medication, comprising:
receiving an order for medication including a delivery location for a user performing the order;
requesting confirmation information from the user;
automatically determining whether order requirements are met;
provisioning the medications for delivery; and
requesting one or more identifiers to complete the delivery to the user.

2. The method of claim 1, further comprising:
ending the transaction in response to determining the order requirements are not met.

3. The method of claim 1, wherein the delivery is performed by an autonomous vehicle.

4. The method of claim 1, wherein the delivery is performed by a delivery vehicle controlled by a delivery person, and wherein the one or more identifiers are communicated from the user to a system associated with the delivery vehicle.

5. The method of claim 1, further comprising:
confirming the delivery location for the medication;
identifying a closest delivery vehicle capable of fulfilling the order;
sending mapping information and delivery information to the delivery vehicle identified;
receiving delivery verification information in response to delivery of the medication.

6. The method of claim 1, further comprising:
electronically processing funds associated with the order.

7. The method of claim 1, wherein the medication is legal marijuana.

8. The method of claim 4, further comprising:
resupplying the delivery vehicle with one or more medications, wherein a secure storage unit for securing the medication.

9. The method of claim 1, wherein the confirmation information specifies an identity, age, and legal status of the user.

10. A system for managing orders of medication, comprising:
a platform managing the orders as received;
one or more databases in communication with the platform storing information associated with the orders; and
one or more networks in communication with the platform for sending and receiving communications associated with the orders, wherein the orders include an order, and wherein the platform receives the order for medication including a location of a user performing the order, requests confirmation information from the user, determines whether order requirements are met, provisions the medications for delivery, and requests one or more identifiers to complete the delivery to the user.

11. The system of claim 10, wherein the platform is one or more servers, and wherein the at least one of the one or more networks represent a cloud network.

12. The system of claim 10, further comprising:
a network of delivery vehicles utilized to fulfill the orders.

13. The system of claim 12, wherein the platform selects one of the network of delivery vehicles capable of delivering the medication.

14. The system of claim 13, wherein the one of the network of delivery vehicles retrieves the medication from a secured location.

15. The system of claim 12, wherein each of the network of delivery vehicles includes a secure storage unit for securing the medication, and wherein the one or more identifiers are required to access the secure storage unit.

16. The system of claim 15, wherein the one or more identifiers include a user identifier and an identifier associated with one of the network of delivery vehicles performing the delivery.

17. A server comprising:
a processor for executing a set of instructions; and
a memory for storing the set of instructions, wherein the set of instructions are executed by the processor to:
receive the order for medication including a delivery location of a user performing the order,
request confirmation information from the user,
determine whether order requirements are met,
provision the medications for delivery, and
request one or more identifiers to complete the delivery to the user.

18. The server of claim 17, wherein the set of instructions are further executed to:
determine the delivery location for the medication;
identify a closest delivery vehicle to the delivery location capable of fulfilling the order;
send mapping information to the delivery vehicle identified; and
receive delivery verification information in response to delivery of the medication.

19. The server of claim 17, wherein the medication is legal marijuana, and wherein the delivery vehicle includes a secure unit for storing the medication until accessed utilizing the one or more identifiers.

20. The server of claim 17, wherein the delivery vehicle is an autonomous vehicle.
